(19) United States
(12) Patent Application Publication
(10) Pub. No.: US 2007/0185496 A1
(43) Pub. Date: Aug. 9, 2007

(54) Device for injecting a viscous material into a hard tissue

(76) Inventors: Lorne Beckman, Montreal (CA); Gamal Baroud, North Hatley (CA); Thomas Steffen, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/506,197

(22) Filed: Aug. 18, 2006

Related U.S. Application Data
(63) Continuation of application No. PCT/CA05/00222, filed on Feb. 18, 2005.

(51) Int. Cl. A61F 2/00

(52) U.S. Cl. 606/93; 606/94; 604/187

(57) ABSTRACT

An injection system for injection of a relatively high viscous material into hard tissue surrounded by soft tissue in an animal body. The injection system comprising an elongated tubular member having a proximal section defining a syringe chamber and a distal section defining a cannula. The cannula having an inside diameter and an outside diameter and a length sufficient for insertion into the hard tissue. The proximal section defining the syringe chamber having an inside diameter greater than the inside diameter of the cannula, the proximal section adapted to be inserted in the soft tissue. The cannula having a bore communicating with the syringe chamber. The injection system comprising a syringe fitted for insertion within the syringe chamber of the proximal section. The syringe having an open end for communicating with the bore of the cannula. The syringe adapted for containing the high viscous material to be injected through the open end and the bore into the hard tissue respectively whereby the injection of the high viscous material is facilitated.

### CROSS-REFERENCE TO RELATED APPLICATIONS

This is a continuation of International Application No. PCT/CA2005/000222 filed Feb. 18, 2005, designating the United States, which itself claims priority on U.S. provisional application 60/545,282 which was filed Feb. 18, 2004, the specifications of both of which are incorporated herein by reference.

### BACKGROUND OF THE INVENTION

The present invention relates to the field of injection biomechanics, and more particularly to a device for injecting a relatively high viscous material into a hard tissue.

Osteoporosis is caused by a gradual loss of bone minerals along with a progressive structural change of trabecular bone (increased porosity, loss of horizontal struts, etc.). Trabecular bone, therefore, loses density and strength and becomes more susceptible to so-called fragility fractures. Vertebral fragility fractures often occur, resulting in chronic pain, progressive deformity and possibly even neurological deficit or damage.

Percutaneous vertebroplasty is an emerging procedure used to strengthen mechanically incompetent vertebrae affected by osteoporosis. This procedure involves injection of a viscous bone cement into the trabecular bone of the vertebra. The cement, once hardened, becomes a permanent reinforcement of the vertebral body and usually drastically diminishes the pain experienced by the patient.

Most often a posterior percutaneous and transpedicular approach is used to access the vertebral body. The approach can be uni- or bipedicular. Alternative surgical approaches are posterolateral and intertransverse, with a direct lateral penetration of the vertebral body.

Percutaneous vertebroplasty has also been used to reinforce vertebral bodies weakened because of osteolytic spinal tumors (hemangioma, metastatic spinal tumors, etc.).

Percutaneous transpedicular vertebroplasty is generally performed with a approximately 15 centimeter long 8 gauge or 11 gauge Jamshidi bone biopsy needle, composed of a straight cannula with a T-handle and removable trocar. The trocar is used along with the cannula to pierce the cutaneous layers and the cortical bone of the vertebra so that the tip of the cannula can be positioned transpedicularly in the cancellous bone of the vertebral body. The trocar is then removed and bone cement is delivered through the cannula, usually under fluoroscopic guidance, into the trabecular bone of the vertebral body.

In order to uniformly infiltrate the vertebral body and avoid unwanted leakage, the bone cement needs to have a viscosity preferably more than 100 Ps*s, possibly even 300 Ps*s. Injecting low viscosity bone cement can cause cement leakage into the surrounding blood vessels, requiring immediate abortion of the procedure to avoid potentially serious complications (e.g., blood pressure drop, lung embolism, death).

Using a high viscosity cement implies that the majority of the injection pressure generated by the surgeon is required to overcome the friction of the cement in the cannula. The required injection pressure can easily reach 1900 KPa in the case of a 15 centimeter long 8 gauge cannula, and up to 6900 KPa in the case of a 15 centimeter 11 gauge cannula, which is well beyond the limit of what the surgeon can manually generate to inject cement with a standard syringe. Pressures generated one-handed with a standard 2 cc syringe have demonstrated maximum obtained pressures in the order of 1700 KPa.

Consequently, the procedure may have to be abandoned because the required injection pressure to continue the injection has become too high to be manually applied. Sometimes a pressure applicator using mechanical advantage is used to overcome this limit. However, a high injection pressure increases the risk of separation of the cement into two phases (i.e., liquid and powder) while the high pressure applicator reduces the tactile feedback of the surgeon and limits the surgeon's ability to accurately control cement flow.

To reduce the required penetration force it was suggested to use a cannula having a reduced tip external diameter as described in U.S. Application Publication No. US2002/0188300A1 by Arramon et al. However, this type of cannula does not address the injection pressure limitations that exist, since the proposed system requires a high pressure applicator generating pressures up to 4000 psi (over 27500 KPa).

In order to limit the risk of damage to the surrounding tissue, U.S. Pat. No. 6,033,411 teaches a stylet with various penetration devices allowing a more controlled penetration of the vertebra. A step in the cannula diameter is described to provide a positive stop to avoid, during the forced insertion, uncontrolled slippage of the cannula through the vertebral body, thereby risking injury to important structures (aorta and v. cava) anterior to the spine. Also, U.S. Patent Application Publication No. US2002/0099384 A1 teaches improvements in the field of staged cannulas. While the patents also suggest a cannula geometry to reduce the pressure requirements, the injection pressures obtained are still well over the limit of what a surgeon can generate manually using a syringe.

Accordingly, there is a need for a device for injecting a relatively high viscous material that addresses some or all of the aforementioned problems.

### SUMMARY OF INVENTION

It is therefore an aim of the present invention to provide a device for injecting a relatively high viscous material into hard tissue surrounded by soft tissue in an animal body whereby the injection of the high viscous material is facilitated.

Therefore, in accordance with the present invention, there is provided a device for injecting a relatively high viscous material into hard tissue surrounded by soft tissue in an animal body. The device comprising an elongated tubular member having a proximal section defining a syringe chamber and a distal section defining a cannula, the cannula having an inside diameter, and an outside diameter and a length sufficient for insertion into the hard tissue, the proximal section defining the syringe chamber having an inside diameter greater than the inside diameter of the cannula, the proximal section adapted to be inserted in the soft tissue, the cannula having a bore communicating with the syringe chamber, the syringe chamber adapted to receive a syringe containing the high viscous material for injection through the bore into the hard tissue whereby injection of the high viscous material is facilitated.

The word “cannula” in the context of this application is defined as a small tube for insertion into a body cavity or into a duct or vessel.

Further in accordance with the present invention, there is provided an injection system for injection of a relatively high viscous material into hard tissue surrounded by soft tissue in an animal body. The injection system comprising an elongated tubular member having a proximal section defining a syringe chamber and a distal section defining a cannula, the cannula having an inside diameter and an outside diameter and a length sufficient for insertion into the hard tissue, the proximal section defining the syringe chamber having an inside diameter greater than the inside diameter of the cannula, the proximal section adapted to be inserted in the soft tissue, the cannula having a bore communicating with the syringe chamber, and a syringe fitted for insertion within the syringe chamber of the proximal section, the syringe having an open end for communicating with the bore of the cannula, the syringe adapted for containing the high viscous material to be injected through the open end and the bore into the hard tissue respectively whereby injection of the high viscous material is facilitated.

Still in accordance with the present invention, there is provided a method for injecting a viscous material through hard tissue surrounded by soft tissue in an animal body. The method comprising the steps of providing an elongated tubular member comprising a distal section defining a cannula adapted to be inserted into hard tissue with minimal damage, and a proximal section defining a syringe chamber having larger outer and inner diameters than the cannula, inserting the cannula into the hard tissue, so that the proximal section remains outside of the hard tissue but at least partially within the soft tissue, inserting a syringe pre-filled with the viscous material into the syringe chamber, and using the syringe to inject the viscous material into the hard tissue through the cannula.

### BRIEF DESCRIPTION OF THE DRAWINGS

Reference will now be made to the accompanying drawings, showing by way of illustration preferred embodiments thereof and in which:

FIG. 1 is a sectional view of a transpedicular injection using an injection system in accordance with a preferred embodiment of the present invention;

FIG. 2 is a sectional view of an elongated tubular member of the injection system in accordance with FIG. 1;

FIG. 3 is a sectional view of the elongated tubular member in accordance with an alternative embodiment of the present invention;

FIG. 4 is a sectional view of a syringe in accordance with a preferred embodiment of the present invention;

FIG. 5 is a sectional view of the syringe inserted in the elongated tubular member of FIG. 2;

FIG. 6 is a sectional view of a telescopic syringe in accordance with an alternative embodiment of the present invention; and

FIG. 7 is a graphical representation of the relative reduction of an injection pressure for various devices having a relative increase of a proximal section inner diameter in accordance with the present invention.

### DETAILED DESCRIPTION OF THE PREFERRED EMBODIMENTS

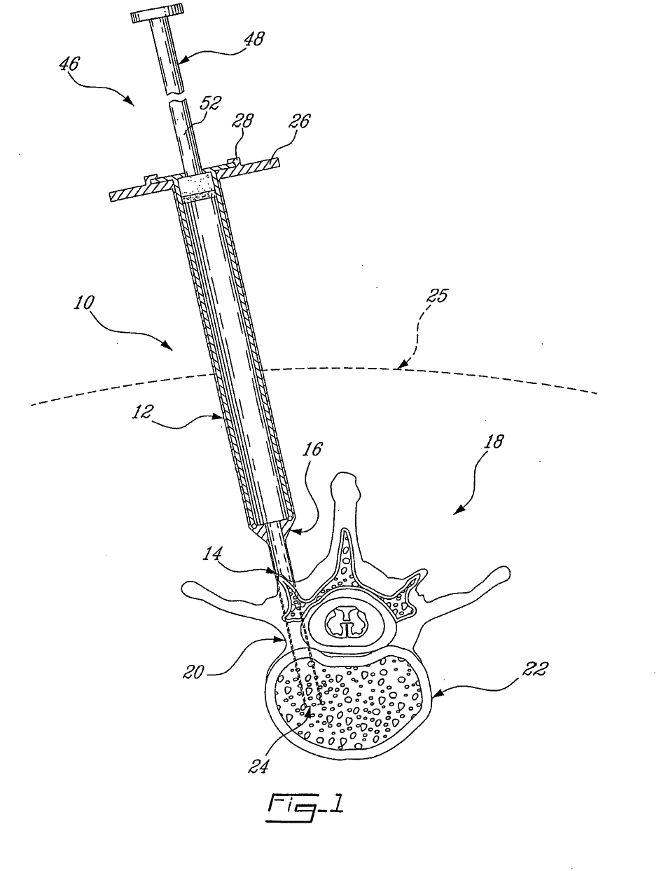

Referring to FIG. 1, an injection system is shown. An elongated tubular member 10 is shown comprising a proximal section defining a syringe chamber 12 and a distal section defining a cannula 14. Preferably also included is a transition section 16 between the syringe chamber 12 and the cannula 14. The elongated tubular member 10 is a device for injecting a relatively high viscous material into hard tissue surrounded by soft tissue in an animal body. More specifically, the elongated tubular member 10 is used in the field of vertebroplasty to inject viscous bone cement into a vertebra 18. In the case of a transpedicular approach, the cannula 14 is inserted through the pedicle 20 of the vertebra 18 and into the vertebral body 22 so as to inject bone cement into the cancellous bone thereof as shown in FIG. 1.

The elongated tubular member 10 may be made entirely of stainless steel or preferably with the syringe chamber constructed of a hard plastic such as a polycarbonate to afford an unobstructed fluoroscopic image.

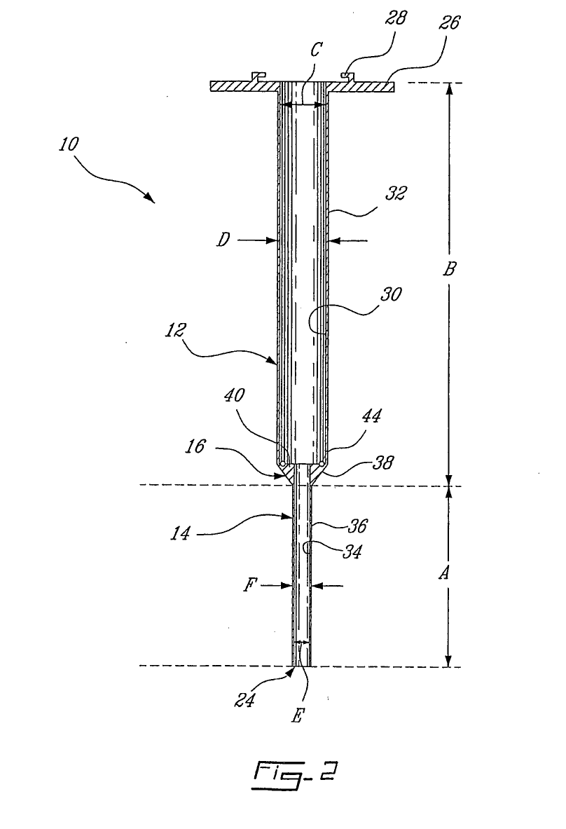

Referring to FIGS. 1 and 2, the cannula 14 has a length A slightly longer than the distance between the point of entry of the tip 24 thereof in the pedicle 20 of a vertebra 18 and the final position of the tip 24 before injection of viscous material in the hard tissue (cancellous bone) of the vertebra 18. The syringe chamber 12 and the transition section 16 are at least partially below the skin 25 level within the soft tissue but outside of the pedicle 20 during the vertebroplasty procedure.

A preferred value for the length A of the cannula 14 is 40 mm, but could vary from 20 mm to 60 mm. A preferred value for the length B of the syringe chamber 12 is 80 mm, but could vary from 60 mm to 140 mm. Therefore, the overall length of the elongated tubular member 10, which corresponds to the sum of A and B, is between 80 and 200 mm.

Referring to FIG. 1, the syringe chamber 12 includes at its proximal end a handle 26 that is of sufficient size and strength to accommodate the twisting and pushing forces exerted by the surgeon during insertion of the cannula 14 through the pedicle 20, and the syringe chamber 12 through the skin 25 and soft tissue layers. Incorporated into the handle 26 is a pair of “L-shaped” tangs 28, the purpose of which will be described later on.

Referring to FIG. 2, the syringe chamber 12 comprises an inner surface 30 defining an inner diameter C and an outer surface 32 defining an outer diameter D. Preferably, the inner diameter C is 7.5 mm, but could vary between 5 and 10.5 mm, and the outer diameter D is 8 mm, but could vary between 5.5 and 11 mm.

The cannula 14 comprises an inner surface 34 defining an inner diameter E and an outer surface 36 defining an outer diameter F. Preferably, the inner diameter E is between 2 and 4 mm and the outer diameter F is 0.4 to 1.0 mm greater than the inner diameter E in order to obtain a cannula 14 wall thickness of at least 0.2 mm. A minimum wall thickness is required so that the cannula 14 can resist the forces applied during bone penetration. Notably, a larger outer diameter F, could preclude the possibility of safely inserting the cannula 14 through the pedicle 20 of a vertebra 18. Preferably, the cannula 14 is designed to be similar in dimensions, materials and design to the standard “Jamshidi bone biopsy needle” commonly used for the procedure.

The transition section 16 acts to link the inner and outer surfaces 30, 32, 34, 36 of the syringe chamber 12 and the cannula 14 respectively. Therefore, the syringe chamber 12 and the cannula 14 are in fluid communication through transition section 16. The outer surface 38 of the transition section 16 is preferably “cone shaped” linking outer surfaces 32 and 36. The inner surfaces 30 and 34 of the syringe chamber 12 and the cannula 12, however, are perpendicularly joined at an annular step 40.

The annular step 40 comprises a circular groove 42 receiving an O-ring 44 for sealing purposes, as will be described further on. Preferably, the O-ring 44 has an outer diameter of approximately 7.5 mm and a section diameter of approximately 1 mm.

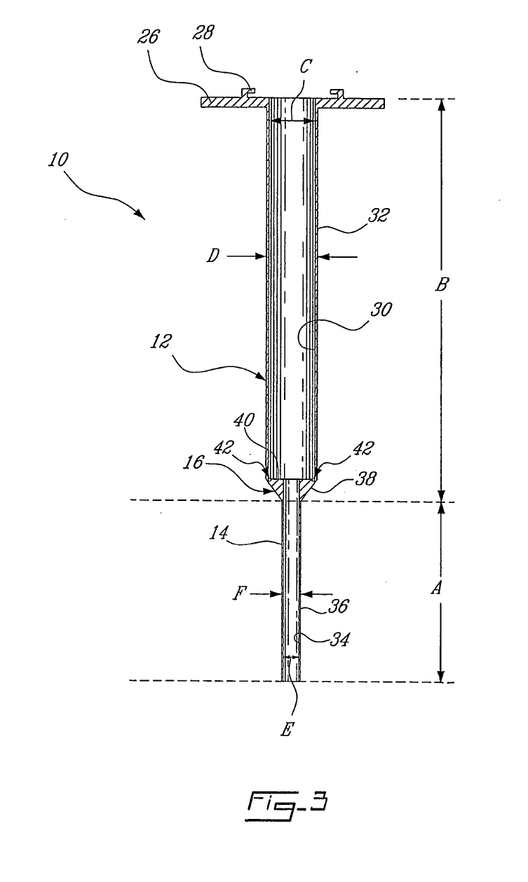

Referring to FIG. 3, an alternative embodiment of the elongated tubular member 10 is shown whereby the O-ring 44 is excluded from the transition section 16.

The preferred dimensions given for the syringe chamber 12 and the cannula 14 are most adapted for the injection of bone cement in a lumbar vertebral body, which usually requires up to 8 cc of bone cement. Other preferred dimensions best suited for thoracic vertebral bodies, which can require up to 6 cc of bone cement, would be a syringe chamber outer diameter D of 7 mm, a syringe chamber inner diameter C of 6.5 mm. Also, the length of the elongated tubular member 10, represented by A+B, would preferably be slightly shorter.

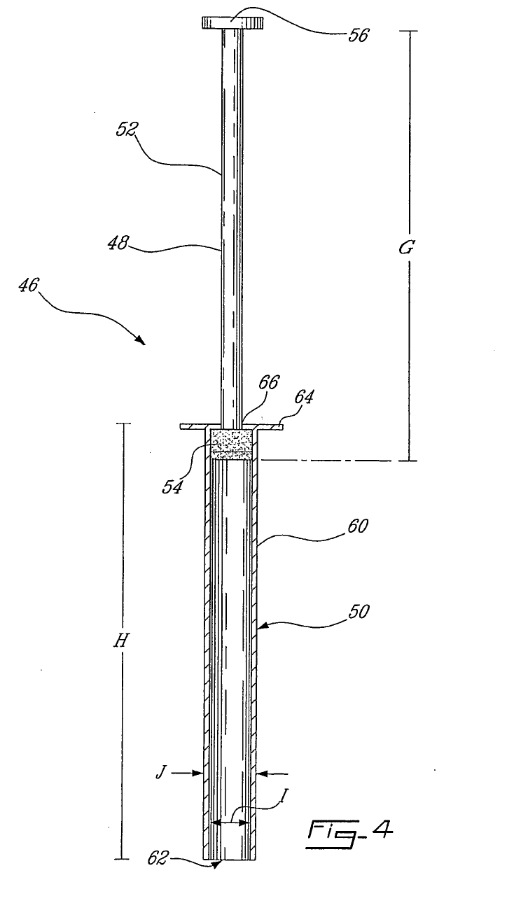

The elongated tubular member 10 is preferably used in conjunction with an open-mouth syringe 46, shown in FIG. 4. The open-mouth syringe 46 comprises two main parts: a plunger 48 and a tube 50.

Referring to FIG. 4, the plunger 48 comprises a rod 52 with a head 54 at a distal end thereof and a knob 56 at a proximal end thereof. The plunger 48 must be sufficiently strong to not fail under the excessive force required for delivering the thick cement. Therefore, the plunger 48 should be made of a robust material, such as a polycarbonate, with an apt choice of dimensions.

Additionally, the head 54 of the plunger 48 should preferably be made from a stiff material such as Teflon to not deform under the high injection forces and thereby increase the frictional forces between the head 54 and the syringe 46 inner surface. Thus, the head 54 is faced with a Teflon, rubber or other suitable material head seal 58 to create a seal inside the tube 50, while allowing the plunger 48 to slide therewithin. Notably, the head seal 58 preferably has an outer diameter suitable for creating a slidable seal within the tube 50.

The knob 56 is of a suitable size and shape to nestle comfortably at the base of the surgeon's palm. In operation, the rod 52 transmits a force applied at the knob 56 to the plunger head 54. The plunger 48 preferably has an overall length G of approximately 100 mm, but could vary between 70 and 160 mm.

Still referring to FIG. 4, the tube 50 comprises a tubular section 60 which, in any case, is configured to be fitted within the syringe chamber 12. In the case of vertebroplasty on the lumbar vertebrae, the tube preferably has an inner diameter I of approximately 7 mm and an out diameter J of approximately 7.5 mm. In the case of vertebroplasty on the thoracic vertebrae, the preferred values for I and J are 6 mm and 6.5 mm respectively.

Furthermore, the tube 50 has a length H, which corresponds to the length B of the syringe chamber 12, whereby the distal tip 62 of the tubular section 60 either slightly compresses the O-ring 44 in FIG. 2, or alternatively extends exactly to the bottom of the circular groove 42 in FIG. 3. In the latter case, the wall thickness of the tubular section 60 would be the same as the section width of the circular groove 42.

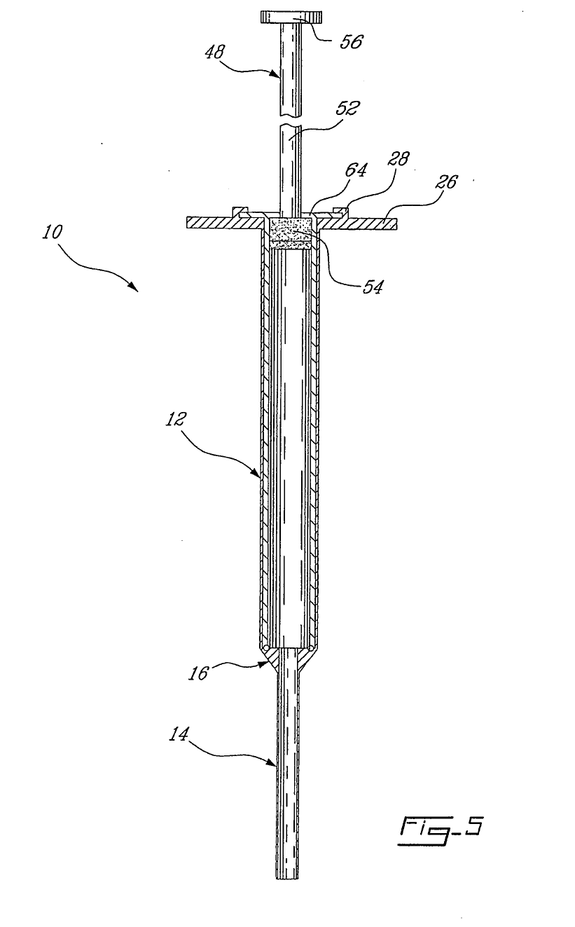

Referring concurrently to FIGS. 4 and 5, the tube 50 further comprises at its proximal end an external tang 64 which is preferably rectangular. The external tang 64 engages with the L-shaped tangs 28 of the cannula 14 (see FIG. 5) in a manner similar to standard “quarter-turn” fasteners so as to lock the tube 50 within the syringe chamber 12. Locking the tube 50 to the syringe chamber 12 presses and seals the distal tip 62 of the tubular section 60 against the O-ring 44, or in the alternative embodiment, within the circular groove 42.

The external tang 64 partially covers the proximal portion of the tubular section 60 such that an opening 66 of approximately the same diameter as the rod 52 of the plunger 48 is defined. The external tang 64 serves as a guide to the rod 52 and also a stop when filling the syringe 46.

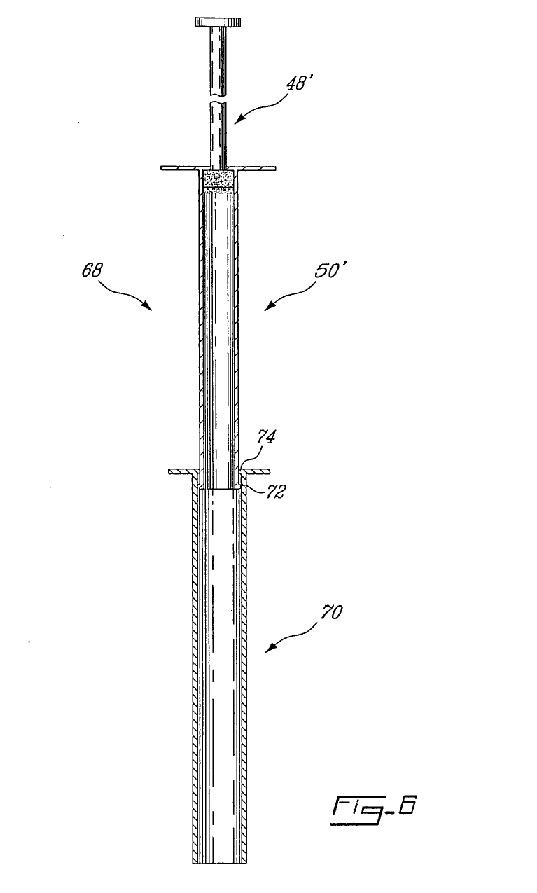

Referring now to FIG. 6, as an alternative embodiment, the elongated tubular member 10 may also be used in conjunction with an open-mouth telescopic syringe 68. The telescopic syringe 66 comprises three main parts, two of which are the same as for the open-mouth syringe 46 therefore similar reference numerals will be used to describe similar parts. The three main parts are: a plunger 48′, a proximal tube 50′, and a distal tube 70.

In this particular embodiment the proximal tube 50′ comprises a sealing ring 72 affixed to the most distal end of the tube 50′. The sealing ring 72 is designed to seal the proximal tube 50′ inside the distal tube 70 while allowing the proximal tube 50′ to slide therewithin. Preferably, the sealing ring 72 is circular and made of rubber, Teflon or an equivalent. The sealing ring 72 also creates a lip that serves as a positive stop at full extension of the proximal tube 50′ in the distal tube 70.

At the proximal end of the distal tube 70 is an internal lip 74 designed to engage the opposite lip created by the sealing ring 72 of the proximal tube 50′ in order to prevent the proximal tube 50′ from completely withdrawing from the distal tube 70.

Notably, in the case of the telescopic syringe 68, it is the distal tube 70 that locks onto the L-shaped tangs 28 of the syringe chamber 12 by way of an external tang 64′ as aforementioned.

It should be understood that the two stage design of the telescopic syringe 68 may be expanded to three or more stages. Each stage can be designed to increase the total volume, or reduce the diameter of the plunger required to generate pressure, or both.

Now referring concurrently to FIGS. 1,2,4, and 5, a transpedicular approach of injecting bone cement into a vertebral body 22 by way of the cannula 14, syringe chamber 12 and open-mouth syringe 46 of the present invention is illustrated. Prior to using the elongated tubular member 10, a trocar is inserted through the skin, through the pedicle 20, and into a vertebral body 22 in a manner known in the art, so that the tip of the trocar is correctly positioned in the cancellous bone. Then, using the trocar as a guide, the elongated tubular member 10 is slid over the trocar through the skin 25 level and soft tissue until the tip 24 of the cannula 14 is positioned correctly. The syringe chamber 12 passes through the skin 25 level and soft tissue and remains outside the pedicle 20.

Once the elongated tubular member 10 is correctly placed, the trocar is carefully removed. Then the open-mouth syringe 46, already filled with bone cement, is inserted into the full depth of the syringe chamber 12 and locked in place by engaging the external tang 64 in the “L-shaped” tangs 28, as explained above.

The bone cement is then injected in the bone following a method known in the art. Basically, the method entails delivering the bone cement by applying pressure to the syringe 46 such that the bone cement exits the tube 50, passes through the transition section 16 and through the cannula 14 into the vertebral body 22. Examples of bone cement that can be used are polymethyl-methacrylate (PMMA) and calcium-phosphate (CaP).

By comparing a standard cannula with a length of up to 150 mm versus the length of approximately 40 mm described herein, the pressure required to inject the bone cement into the cancellous bone is decreased by a factor of three to four. The cannula and syringe chamber exemplified herein require an injection pressure as low as 700 KPa, which is well below the physical limit of the physician of about 1700 KPa. Therefore, the physician can manually perform the injection more easily while using a bone cement with a high viscosity. Alternatively, the physician can manually perform the injection using a bone cement of an even higher viscosity while still staying within the 1700 KPa limit. Notably, the use of a cement of high viscosity maximizes cement infiltration in the bone and diminishes the risks of unwanted and dangerous cement leakage through blood vessels.

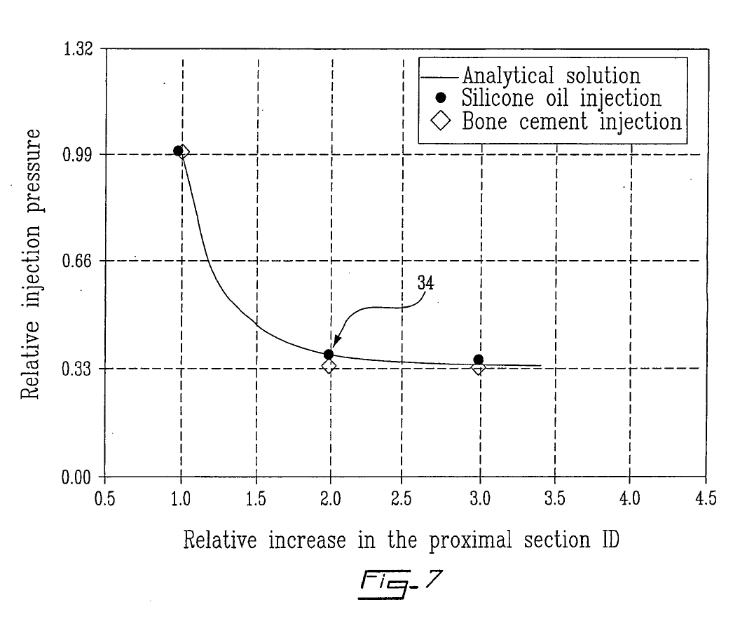

Now referring to FIG. 7, the graphical representation of the relative reduction of an injection pressure for various devices having a relative increase of a proximal section inner diameter will be explained by way of experimental data.

### Experiment A

Method

Three different elongated tubular members were tested. A first tubular member was a traditional single stage inner diameter 8 gauge cannula (inner diameter of 3.125 mm, length of 120 mm). Second and third tubular members were designed according to the present invention having a staged inner diameter as shown in FIG. 1. Thus, the distal end defining the cannula having an inner diameter E equal to the inner diameter of the 8-gauge cannula, i.e. 3.125 mm. The syringe chamber 12 having a length B of 80 mm and a cannula 14 length A of 40 mm. The second tubular member featured a syringe chamber 12 inner diameter C of approximately 6.35 mm for a relative increase of about 2 or 200% compared to the inner diameter E of the cannula thereof. The third tubular member featured a syringe chamber inner diameter C of approximately 9.525 mm for a relative increase of approximately 3 or 300% compared to the inner diameter E of the cannula thereof.

A thick-viscous silicone oil of 95 Ps·s (Viscosity standard 100000, Brookfield Engineering, Middleboro, Mass.) was first injected through the three elongated tubular members. The silicone oil has a viscosity similar to bone cement while allowing the effect of the tubular member geometry to be isolated from the effect of the material, since the viscosity of silicone oil is Newtonian and less sensitive to changes in temperature than the viscosity is of bone cement.

A bone cement was also injected through the elongated tubular members. The bone cement used was an acrylic polymer that is mainly used in dental and research laboratories, DP-Pour acrylic cement (DenPlus Inc., Montreal, Canada). DP-Pour has a composition and rheological behavior similar to that of bone cements used in vertebroplasty. The bone cement was mixed with a liquid-to-powder ratio in accordance with the recommendation of the manufacturers, namely 18.0 mL of liquid with 30.8 g of powder. A spatula was used to mix the bone cement at approximately 60 beats per minute for approximately 50 seconds until the powder was visually dissolved in the liquid. To match clinical conditions, the DP-Pour was injected after it obtained a dough-like consistency, which was approximately 11 minutes after the liquid was added to the powder.

For injection, a 20-cc syringe was filled with silicone oil or DP-Pour and connected to the elongated tubular member tested. The tubular member and the syringe were attached to a servo-hydraulic testing machine (Mini Bionix 856, MTS, Eden Prairie, Minn.) and the plunger of the syringe was displaced at the constant injection rate of approximately 4 cc/min, which is representative of a clinical situation.

For each experiment, the injection pressure was recorded. The reliability of the injection pressure measurements was confirmed by comparing the measurements to an analytical solution. The injection pressure, ΔPinj, can be estimated using Hagen-Poisseuille's law:

$$Δ {P}_{inj} = Q \frac{8 η}{π} \frac{L}{{a}^{4}} \qquad ( 1 )$$

where a and L are the cannula radius and length, η is the cement viscosity, and Q is the volume flow rate. According to equation (1), the injection pressure decreases in an over-proportional fashion (a⁴) with an increase of the radius of the proximal section and increases in a linear fashion with respect to the length of the cannula.

Results

The results of the experiments with both injection materials are shown and compared with the analytical solution in FIG. 7. The experimental results of both the silicone oil and the bone cement are consistent with the analytical solution. The results show that the injection pressure of the second tubular member is reduced to about 0.33 of 33% of the injection pressure of the first cannula. Therefore, augmenting the inner diameter of the proximal section by a factor of 2 brings a considerable reduction in injection pressure. However, increasing the inner diameter of the proximal section further does not seem to bring a significant further injection pressure reduction. This is shown in the case of the third tubular member, having a proximal inner diameter about three times the size of the inner diameter of the cannula, where the injection pressure is also reduced to about 0.33 or 33% of the injection pressure of the first single stage tubular member.

The analytical and experimental findings are consistent. Therefore, it appears that the proposed tubular member design reduces the injection pressure significantly. However, it appears that after a leveling point 34 (see FIG. 7), the reduction of pressure brought by the increase in the syringe chamber inner diameter is minimal.

Furthermore, this experiment has shown that cement behaviour is close to Newtonian fluid behaviour; therefore it can be predicted that increased or decreased proximal section inner diameters and/or lengths would fall under Hagen-Poisseuille's Law for injection pressures.

The above description is meant to be exemplary only, and one skilled in the art will recognize that changes may be made to the embodiments described without department from the scope of the invention disclosed. For example, the primary application of the elongated tubular member for injecting bone cement for mechanical augmentation of osteoporosis-induced thinned out bone described herein is for vertebroplasty, but the technique may also be used to mechanically augment bones in other body locations (e.g., proximal femur, distal radius, etc.) Still other modifications which fall within the scope of the present invention will be apparent to those skilled in the art, in light of a review of this disclosure, and such modifications are intended to fall within the appended claims.

What is claimed is:

1. A device for injecting a relatively high viscous material into hard tissue surrounded by soft tissue in an animal body, the device comprising:
an elongated tubular member having a proximal section defining a syringe chamber and a distal section defining a cannula, the cannula having an inside diameter, and an outside diameter and a length sufficient for insertion into the hard tissue, the proximal section defining the syringe chamber having an inside diameter greater than the inside diameter of the cannula, the proximal section adapted to be inserted in the soft tissue, the cannula having a bore communicating with the syringe chamber, the syringe chamber adapted to receive a syringe containing the high viscous material for injection through the bore into the hard tissue whereby injection of the high viscous material is facilitated.

2. The device according to claim 1, wherein the inside diameter of the cannula ranges between 2 and 4 mm.

3. The device according to claim 1, wherein the inside diameter of the syringe chamber defined by the proximal section ranges between 5 and 10.5 mm.

4. The device according to claim 1, wherein the syringe chamber has an outside diameter greater than the outside diameter of the cannula.

5. The device according to claim 4, wherein the outside diameter of the cannula is 0.4 to 1.0 mm greater than the inside diameter of the cannula in order to obtain a cannula wall thickness of between 0.2 to 0.5 mm.

6. The device according to claim 1 wherein the elongated tubular member further comprises a transition section disposed between the syringe chamber and the cannula providing communication between the bore and the syringe chamber.

7. The device according to claim 6, wherein the transition section has an outside surface in the shape of a cone.

8. The device according to claim 7, wherein the transition section has an inner surface defining an annular step perpendicularly joining the inside diameter of the cannula and of the syringe chamber.

9. The device according to claim 8, wherein the annular step comprises a circular groove.

10. The device according to claim 9, wherein the circular groove is adapted to receive an open end of the syringe.

11. The device according to claim 9, wherein the circular groove contains an O-ring for sealing purposes.

12. The device according to claim 1, wherein the syringe received in the syringe chamber is a telescopic syringe.

13. The device according to claim 1, wherein the proximal portion includes a locking means for engagement with the syringe to lock the syringe thereto.

14. The device according to claim 1, wherein the high viscosity material is injected into the hard tissue with a delivery pressure lower than 1700 KPa.

15. The device according to claim 1, wherein the viscous material is bone cement.

16. The device according to claim 1, wherein the proximal section is generally twice as long as the distal section.

17. The device according to claim 1, wherein the inner diameter of the proximal section is substantially twice the inside diameter of the cannula so as to optimize the relationship between the injection pressure and the inside diameters.

18. An injection system for injection of a relatively high viscous material into hard tissue surrounded by soft tissue in an animal body, the injection system comprising:
an elongated tubular member having a proximal section defining a syringe chamber and a distal section defining a cannula, the cannula having an inside diameter and an outside diameter and a length sufficient for insertion into the hard tissue, the proximal section defining the syringe chamber having an inside diameter greater than the inside diameter of the cannula, the proximal section adapted to be inserted in the soft tissue, the cannula having a bore communicating with the syringe chamber; and
a syringe fitted for insertion within the syringe chamber of the proximal section, the syringe having an open end for communicating with the bore of the cannula, the syringe adapted for containing the high viscous material to be injected through the open end and the bore into the hard tissue respectively whereby injection of the high viscous material is facilitated.

19. The injection system according to claim 17, wherein the syringe is a telescopic syringe.

20. The injection system according to claim 18, wherein the inside diameter of the proximal section is substantially twice the inside diameter of the cannula thereby optimizing the relationship between the injection pressure and the inside diameters.

21. A method for injecting a viscous material through hard tissue surrounded by soft tissue in an animal body, the method comprising the steps of:
providing an elongated tubular member comprising a distal section defining a cannula adapted to be inserted into hard tissue with minimal damage, and a proximal section defining a syringe chamber having larger outer and inner diameters than the cannula;
inserting the cannula into the hard tissue, so that the proximal section remains outside of the hard tissue but at least partially within the soft tissue;
inserting a syringe pre-filled with the viscous material into the syringe chamber; and
using the syringe to inject the viscous material into the hard tissue through the cannula.

22. The method according to claim 21, wherein the viscous material is injected with a delivery pressure lower than 1700 KPa.

23. The method according to claim 21, wherein the inside diameter of the proximal section is substantially twice the inside diameter of the cannula thereby optimizing the relationship between the injection pressure and the inside diameters.
